(19) United States
(12) Patent Application Publication
(10) Pub. No.: US 2024/0350480 A1
(43) Pub. Date: Oct. 24, 2024

(54) STABLE PHARMACEUTICAL COMPOSITION OF METHYLNALTREXONE FOR AN ORAL ADMINISTRATION

(71) Applicant: Lupin Limited, Mumbai (IN)

(72) Inventors: Brijesh Purohit, Pune (IN); Anirudha Bhagirath Kute, Pune (IN); Sajeev Chandran, Pune (IN); Makarand Krishnakumar Avachat, Pune (IN); Ashish Deshmukh, Pune (IN)

(73) Assignee: Lupin Limited, Mumbai (IN)

(21) Appl. No.: 18/639,693

(22) Filed: Apr. 18, 2024

(30) Foreign Application Priority Data
Apr. 18, 2023 (IN) 202321028283

(51) Int. Cl. A61K 31/485; A61K 9/20; A61K 31/795

(52) U.S. Cl. CPC A61K 31/485; A61K 9/2013; A61K 9/2027; A61K 9/2054; A61K 31/795

(57) ABSTRACT

The present invention relates to a stable pharmaceutical composition comprising methylnaltrexone and its pharmaceutically acceptable salts. The invention further relates to compositions comprising of methylnaltrexone and its pharmaceutically acceptable salts and ion exchange resin and its process of preparation. The compositions are substantially free of impurities. The compositions provide the desired immediate release of methylnaltrexone and were found to be stable under accelerated stability conditions. The compositions are administered orally once daily for the treatment of opioid-induced constipation in adult patients with cancer and chronic non-cancer pain.

### FIELD OF INVENTION

The present invention relates to solid oral pharmaceutical compositions of methylnaltrexone or a pharmaceutically acceptable salt thereof. The present invention also relates to a process of preparing and using said compositions for treatment of opioid-induced constipation (OIC) in adult patients with cancer and chronic non-cancer pain. More particularly, the invention relates to immediate release composition of methylnaltrexone or a pharmaceutically acceptable salt thereof complexed with ion exchange resin.

### BACKGROUND

Opioids are highly administered for the management of acute and chronic pain. Opioid usage for the management of pain in cancer and non-cancer patients is associated with several centrally and peripherally mediated adverse effects. Peripheral mu-opioid receptors on the gastrointestinal (GI) tract are responsible for opioid-induced bowel dysfunctions (OIBD). By affecting the transit time of the GI tract, opioids cause constipation. Constipation occurs in approximately 40-90% of individuals taking opioids for pain control. Extensive prevalence of opioid-induced constipation (OIC) experienced by patients is one of the major causes of discontinuing the analgesic treatment. Therefore, non-compliance and avoiding opioid therapy may occur due to these adverse effects. Quality of life (QOL) is significantly impaired in patients with OIC. Both mental and physical components in these patients are affected. On the other hand, discontinuation of opioid treatment due to OIC results in uncontrolled pain that affects QOL.

Peripheral mu-opioid receptor antagonists (PAMORAs) are successful options in OIC treatment. Methylnaltrexone and its salts block the mu-opioid receptor in the GI tract, without affecting the central opioid-induced analgesia. It is the first PAMORA approved for the OIC in patients with advanced illness when laxatives were not efficient. Methylnaltrexone, a quaternary derivative of naltrexone, is a peripherally nonselective mu-opioid receptor antagonist which can provide causal treatment for opioid-induced constipation (OIC), yet not interfere with the analgesic effect. Having the general structure of a quaternary ammonium salt, Methylnaltrexone is a compound with greater polarity and lower lipid solubility. Therefore, methylnaltrexone has restricted access to the blood-brain barrier and decreases the constipating effects of opioid pain medications. However, as a hydrophilic compound, methylnaltrexone has limited gastrointestinal absorption (Yuan, C. S et al, 1997 and Becker G et al, 2007). Due to positive charge of the quaternary amine, methylnaltrexone is poorly absorbed in the gastrointestinal tract. In general, on oral administration, less than about 5% of methylnaltrexone is absorbed into the bloodstream. The structure of methylnaltrexone is shown below.

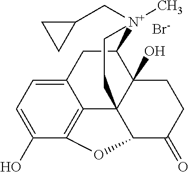 [SMILES: C[N+]1(CC2CC2)CC[C@]23c4c5ccc(O)c4O[C@H]2C(=O)CC[C@@]3(O)[C@H]1C5 ; [Br-]]

It has been used in patients to reduce opioid-induced side effects such as constipation, pruritus, nausea, and urinary retention (see, e.g., U.S. Pat. Nos. 5,972,954, 5,102,887, 4,861,781, and 4,719,215; and Yuan et al., Drug and Alcohol Dependence 1998, 52, 161). The dosage form of methylnaltrexone used most often in these studies has been a solution of methylnaltrexone for intravenous injection. Methylnaltrexone subcutaneous injection Relistor® is available for the treatment of opioid induced constipation in patients with advanced illness receiving palliative care when response to laxative therapy has not been sufficient. The dose of Methylnaltrexone for treating methadone maintenance patients was explored in U.S. Pat. No. 6,559,158.

An oral dosage form that releases certain opioid antagonists such as naloxone, N-methylnaloxone, and N-methylnaltrexone “over the whole gastrointestinal tract is disclosed in U.S. Pat. No. 6,419,959.” Further it states that opioid antagonists are not always suitable for administration in an immediate release form due to dose limiting side effects. To address these issues, the U.S. Pat. No. 6,419,959 suggests dosing certain opioid antagonists, including methylnaltrexone, in a controlled-release dosage form, thereby delivering these antagonists at acceptable doses locally across the entire gastrointestinal tract. However, data with respect to methylnaltrexone specifically was not reported.

In U.S. Pat. No. 6,274,591, it was demonstrated that an enteric coated methylnaltrexone which released substantially no methylnaltrexone in the stomach was more effective than uncoated methylnaltrexone in antagonizing the oral-cecal delay caused by morphine. Hence, U.S. Pat. No. 6,274,591 suggests and claims delivering effective amounts of Methylnaltrexone using an oral dosage that by-passes the stomach altogether.

Immediate release methylnaltrexone is commercially available as Relistor® tablets 150 mg. The film-coated tablet contains 150 mg of methylnaltrexone bromide which is equivalent to 122.5 mg methylnaltrexone.

Patents and publications related to Relistor® tablets are U.S. Pat. Nos. 8,524,276, 8,956,651, 9,314,461, 10,307,417, 10,376,505, 1,037,650, 10,507,206, 20,160,206612, US 20200121673 and US 20130317050, wherein an ion pair between the positively charged methylnaltrexone and a negatively charged moiety was postulated to make a “pair” that is more hydrophobic than methylnaltrexone bromide and thereby enhanced the absorption of Methylnaltrexone in the stomach.

Ion pairing was done using methylnaltrexone and an amphiphilic pharmaceutically acceptable excipient particularly, sodium lauryl sulfate in a solid dosage form, together with a rapid-acting disintegrant e.g., a carbon dioxide generating disintegrant which when dissolved in solution was effective to induce laxation.

It was demonstrated in WO 2023031955, that the composition is free of an ion pairing agent and instead uses a solubilizer such as polysorbate 80 or polyoxyl 40 hydrogenated castor oil that is an alternative to an ion pairing agent which can enhance gastrointestinal absorption of Methylnaltrexone.

In CN 105582011, inventors prepared the stable solid composition of methylnaltrexone bromide with acidic stabilizing agent at weight ratio of 10:1 to 1:10 and assessed the relative bioavailability of the said composition on 6 healthy male dogs and found that 77.87% absolute bioavailability and inventors assessed the effectiveness of the said composition for constipation treatment by using rat constipation model and found to be successful.

In CN 104274414, inventors prepared methylnaltrexone complex with phospholipid, further, this complex can change the determination of oil-water partition coefficient of methylnaltrexone, strengthening fat-soluble, thus improves the problem of oral absorption. Further, inventors also prepared methylnaltrexone oral tablet containing the said complex with stabilizing agent such as organic acid.

Thus, in summation, the prior art either suggests effectiveness of controlled release, enteric release composition of methylnaltrexone or immediate release methylnaltrexone composition having ion pairing agent and/or solubilizing agent and/or stabilizing agent and/or phospholipid for the effective treatment OIC

Therefore, as such there remains a need to develop cost effective, simple, and easily reproducible solid oral composition of methylnaltrexone and the process of preparing such composition for the treatment of OIC.

Ion exchange resins are utilized for achieving simple, reproducible, and cost effective solid oral dosage form. Various attempts have been made to further utilize the technology in several dosage forms as well.

Ion exchange resins are cross-linked water insoluble polymer carrying ionizable functional groups and have received considerable attention from pharmaceutical industry because of their versatile properties as drug delivery vehicle.

Currently, ion exchange resins that are used in pharmaceutical applications serve a variety of functions, e.g., taste masking, improving dissolution of poorly soluble drugs, or improving stability of pharmacologically active drugs.

As such, the instant invention encompasses a pharmaceutical composition comprising methylnaltrexone or a pharmaceutically acceptable salt thereof and ion exchange resin preferably with an immediate release feature that is cost effective and allows a simple and reproducible process of preparation.

### OBJECTIVE OF THE INVENTION

The present invention provides the following aspects, subject matters, and preferred embodiments, which respectively taken alone or in combination, contribute to solving the object of the present invention.

The main objective of the invention is to provide immediate release pharmaceutical compositions comprising methylnaltrexone or a pharmaceutically acceptable salt thereof, a pharmaceutically acceptable ion exchange resin and a pharmaceutically acceptable carrier for the treatment of opioid-induced constipation (OIC).

Another objective of the present invention is to provide immediate release pharmaceutical compositions comprising methylnaltrexone or a pharmaceutically acceptable salt or derivative thereof, a pharmaceutically acceptable ion exchange resin and a pharmaceutically acceptable carrier by avoiding ion pairing agent, stabilizing agent, and solubilizing agent.

It is another objective of the present invention to provide an immediate release pharmaceutical composition comprising methylnaltrexone or a pharmaceutically acceptable salt or derivative thereof, a pharmaceutically acceptable ion exchange resin and a pharmaceutically acceptable carrier wherein the ratio of methylnaltrexone to ion exchange resin ranges from about 1:0.01 to about 1:10.

It is another objective of the present invention to provide pharmaceutical compositions containing from about 50 mg to about 500 mg of methylnaltrexone bromide. Preferably the invention provides pharmaceutical compositions containing from about 150 mg to about 450 mg of methylnaltrexone bromide.

It is yet another objective of the present invention to provide pharmaceutical composition comprising about 450 mg of methylnaltrexone once daily as three tablets containing about 150 mg of methylnaltrexone for the treatment of OIC. Further, it is another objective of the present invention to provide pharmaceutical composition comprising about 150 mg of methylnaltrexone e a day for the treatment of OIC.

It is also an objective of the present invention to develop a process for the preparation of oral stable composition of methylnaltrexone or its pharmaceutically acceptable salt thereof, wherein, said process comprises;
(i) preparing a drug-resin complex comprising methylnaltrexone or a pharmaceutically acceptable salt thereof and a pharmaceutically acceptable ion exchange resins and one or more pharmaceutically acceptable excipients;
(ii) sifting extragranular material and lubricant
(iii) mixing the obtained drug-resin complex with sifted extragranular material and lubricant followed by compression of the obtained blend with suitable tooling.
(iv) applying coating composition on the obtained compressed blend.

### DETAILED DESCRIPTION OF INVENTION

The invention will now be described in detail in connection with certain preferred and optional aspects, so that various aspects thereof may be more fully understood and appreciated.

The present invention relates to a pharmaceutical composition comprising methylnaltrexone or a pharmaceutically acceptable salt or derivative thereof, a pharmaceutically acceptable ion exchange resin and a pharmaceutically acceptable carrier for the treatment of patients suffering from opioid-induced constipation (OIC).

The present invention relates to an immediate release pharmaceutical compositions comprising methylnaltrexone or a pharmaceutically acceptable salt or derivative thereof, a pharmaceutically acceptable ion exchange resin, and a pharmaceutically acceptable carrier.

Particularly, the invention provides an immediate release pharmaceutical compositions comprising methylnaltrexone or a pharmaceutically acceptable salt or derivative thereof, a pharmaceutically acceptable ion exchange resin and a pharmaceutically acceptable carrier by avoiding ion pairing agent, stabilizing agent and a solubilizing agent for the treatment of patients suffering from OIC.

In one embodiment, the present invention relates to an immediate release pharmaceutical composition comprising methylnaltrexone or a pharmaceutically acceptable salt or derivative thereof, a pharmaceutically acceptable ion exchange resin and a pharmaceutically acceptable carrier wherein the ratio of methylnaltrexone to ion exchange resin ranges from about 1:0.01 to about 1:10.

In another embodiment, the pharmaceutical composition comprises about 150 mg of methylnaltrexone, or a salt thereof.

In certain embodiments, the compositions, and formulations thereof, comprise a salt of formula I:

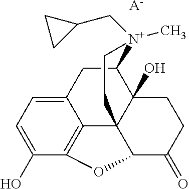

wherein A− is a suitable anion. In certain embodiments, A− is the anion of a Brønsted acid. Exemplary Brønsted acids include hydrogen halides, carboxylic acids, sulfonic acids, sulfuric acid, and phosphoric acid. In certain embodiments, A− is chloride, bromide, iodide, fluoride, sulfate, bisulfate, tartrate, nitrate, citrate, bitartrate, carbonate, phosphate, malate, maleate, fumarate sulfonate, methylsulfonate, formate, carboxylate, sulfate, methylsulfate or succinate salt. In certain embodiments, A− is trifluoroacetate. In certain embodiments, A− is bromide.

In another embodiment, the invention provides pharmaceutical compositions containing from about 50 mg to about 500 mg of methylnaltrexone bromide. Preferably the invention provides pharmaceutical compositions containing from about 150 mg to about 450 mg of methylnaltrexone bromide equivalent to about 122.5 to about 367.5 mg methylnaltrexone.

In another embodiment, the composition comprises orally administering about 150 mg of methylnaltrexone, or a salt thereof. In a related embodiment, about 150 mg of methylnaltrexone is administered as one tablet comprising about 150 mg of methylnaltrexone.

In another embodiment, the composition comprises orally administering about 300 mg of methylnaltrexone, or a salt thereof. In a related embodiment, about 300 mg of methylnaltrexone is administered as two tablets each comprising about 150 mg of methylnaltrexone.

In another embodiment, the composition comprises orally administering about 450 mg of methylnaltrexone, or a salt thereof. In one embodiment, about 450 mg of methylnaltrexone is administered as three tablets each comprising about 150 mg of methylnaltrexone.

In another embodiment, the invention provides a solid oral pharmaceutical composition containing 450 mg of methyl naltrexone bromide in a single unit dosage form.

In one embodiment the compositions of the present invention may be a coated or uncoated tablet, capsule, granule, powder.

In another embodiment, examples of suitable pharmaceutical dosage forms are including but not limited to granules, multiunit particulate systems (MUPS), pellets, spheres, tablets, dispersible tablets, soft capsules, hard capsules, mini-tablets, beads, particles. Preferable dosage forms are tablets or capsules.

In another embodiment, the present invention provides various dosage forms of methylnaltrexone or its salts, which include but are not limited to immediate release formulations, delayed release formulations, multiunit particulate system etc. Yet another embodiment of the present invention provides various processes to prepare oral pharmaceutical compositions of methylnaltrexone or its salts, and these processes include direct compression, wet granulation, dry granulation, spheronization, extrusion and spheronization spray drying or melt extrusion, etc. Additionally, compositions of the present invention can also be consumed as suspensions before processing into tablets or alternatively, fast disintegrating tablets can be added to water to form a fine suspension which can be consumed.

### Definitions

As used herein, the term “opioid induced constipation” (OIC) refers to a subject who suffers from constipation resulting from opioid therapy.

The term “constipation” as used herein, refers to a condition in which a subject suffers from infrequent bowel movements or bowel movements that are painful and/or hard to pass.

The term “ion exchange resin particles”, as used herein, means anionic or cationic ion exchange resins.

The term “methylnaltrexone ion exchange resin complex”, as used herein, means methylnaltrexone containing ion exchange resin particle in which there is an ionic bond between methylnaltrexone and the ion exchange resin particle.

The term “Drug-resin complex”, as used herein, means ion exchange resins that are water-insoluble polymers in the forms of very small particles and beads. The drug is bound to the ion exchange resin by an acid-base reaction. Either a cation or an anion exchange resin can be used, depending on whether the drug to be bound is acidic or basic, i.e., a basic drug is bound to a cation exchange resin and an acid drug is bound to an anion exchange resin.

The term “about”, as used herein, is defined as all numerical values relating to amounts, weights, and the like, wherein each particular value is plus or minus 10%.

The term “diluent” or “filler” is an excipient that adds bulkiness to a pharmaceutical composition.

The term “disintegrant” is an excipient that hydrates a pharmaceutical composition and aids in tablet dispersion.

The term “osmotic agent” is an excipient that increases the osmotic pressure and may be disposed in the water-permeable body or included in the drug formulation.

The term “lubricant” used herein, is a non-toxic excipient that decreases friction between tablet's surface and the die wall cavity in which the tablet was formed and to reduce wear and tear of dies and punches.

The term “coat” or “coating” or “coated” are equivalent terms and refers to film coating on to uncoated tablets or granules or powder.

The term “immediate release” (IR) meaning, for example, a release of at least 60% of the drug under physiological conditions (pH, temperature), such as within 60 minutes or less, such as within 30 or less, or within 20 minutes or less, or within 15 minutes or less. An immediate release drug product is considered rapidly dissolving when no less than 85% of the drug substance dissolves within 30 minutes in a volume of 900 ml or

less in media of varying pH.

The ion exchange resins are basically water-insoluble polymers in the forms of very small particles and beads. The drug is bound to the ion exchange resin by an acid-base reaction. Either a cation or an anion exchange resin can be used, depending on whether the drug to be bound is acidic or basic, i.e., a basic drug is bound to a cation exchange resin and an acid drug is bound to an anion exchange resin.

In one embodiment, any ion exchange resin can be used to prepare the methylnaltrexone-ion exchange resin complex of the present invention, provided it is pharmaceutically acceptable. Ion exchange resins useful in the practice of the present invention include, but are not limited to, anionic resins such as: DUOLITE® AP143/1083 (cholestyramine resin USP) and cationic resins such as AMBERLITE® IRP-64 (a porous copolymer of methacrylic acid crosslinked with divinylbenzene), AMBERLITE® IRP-69 (Sodium polystyrene sulfonate USP) and AMBERLITE® IRP-88. AMBERLITE® IRP 69 is the preferred resin. Further, said AMBERLITE® IRP 69 (sodium polystyrene sulfonate) is available commercially as a sodium salt. However, it is within the scope of the present invention to convert the sodium salt to other salt forms including, but not limited to, K and Li.

In another embodiment, methylnaltrexone or a pharmaceutical salt or derivative thereof complexed with ion exchange resin particles are in a ratio range from 1:0.01 to 1:10.

In another embodiment, the examples of diluent or filler include but not limited to calcium phosphate, dicalcium phosphate, tricalcium phosphate, calcium sulfate, anhydrous lactose, spray dried lactose, hydrated lactose, cellulose, spray dried microcrystalline cellulose, spray dried combinations comprising microcrystalline cellulose and lactose, silicified microcrystalline cellulose, kaolin, bentonite, mannitol, starch, magnesium carbonate, sorbitol, sucrose, inositol, compressible sugar, trehalose and xylitol, and mixtures thereof.

In another embodiment, the composition comprises from about 10 to about 90%, or from about 15 to about 85%, or from about 20 to about 75%, or from about 30 to about 70%, or from about 15 to about 30%, or from about 50 to about 90%, or from about 60 to about 85%, of diluent or filler.

In another embodiment, the examples of disintegrants include but are not limited to crospovidone, sodium croscarmellose and/or sodium starch glycolate, pregelatinized starch, low substituted hydroxypropyl cellulose (L-HPC).

In another embodiment, the composition comprises from about 3 to about 20% of disintegrant, or from about 5 to about 20%, or from about 5 to about 15%, of disintegrant.

In another embodiment, the examples of osmotic agents include are but not limited to sucrose, xylitol, glucose, lactose; salts such as sodium chloride, potassium chloride; low molecular weight hydrophilic polymers such as cellulose ethers, maltodextrins, and cyclodextrins.

In another embodiment, the composition comprises from about 0.1 to about 10% of osmotic agent, or from about 0.1 to about 5%, of osmotic agent.

In another embodiment, the examples of coating excipients include but are not limited to those known in the art, such as cellulose derivatives (hydroxypropyl methylcellulose, hydroxyethylcellulose, hydroxypropyl cellulose and their derivatives), acrylic and methacrylic copolymers of different molecular weights, and mixtures thereof, water-soluble polymers for instance, aminoalkyl methacrylate copolymer E, hypromellose, methyl cellulose, methyl hydroxyethyl cellulose, Opadry, calcium carmellose, sodium carmellose, polyvinyl pyrrolidone, polyvinyl alcohol, dextrin, pullulan, gelatin, agar and gum Arabic, among others. Preferably coating composition comprises Polyvinyl Alcohol, Titanium Dioxide, Talc and Polyethylene glycol. The coating layers over the tablet may be applied as solution/dispersion of coating ingredients using conventional techniques known in the art selected from spray coating in a conventional coating pan or fluidized bed processor, dip coating, and the like. In one embodiment, weight gain after coating is up to about 5%, preferably up to about 3%.

In one embodiment, the examples of lubricant include but are not limited to magnesium stearate, stearic acid, palmitic acid, calcium stearate, talc, carnauba wax and sodium stearyl fumarate. Preferably, the lubricant of the present invention is present in an amount of about 0.1 to about 5% by weight, relative to the weight of the solid formulation.

In another embodiment, methylnaltrexone or a pharmaceutical salt or derivative thereof complexed with ion exchange resin particles are in a ratio range from about 1:0.01 to about 1:10 which is critical for maintaining therapeutic efficacy and bioavailability.

In another embodiment the composition of the present invention also may include a rapid-acting disintegrant, wherein the composition dissolves within about 15 minutes in the stomach. In at least one embodiment, at least 50% of the methylnaltrexone in the composition is dissolved in 15 minutes.

In another embodiment, at least 60%, 75%, 80%, 85%, 90%, 95%, or even 99% of the methylnaltrexone in the present composition is dissolved in 15 minutes.

In another embodiment, the methylnaltrexone in the present composition can dissolve within 10 minutes or even within 5 minutes.

Yet in another embodiment, the dissolution of the present compositions may be simulated by in vitro studies in a dissolution apparatus with paddles at 100 rpm in 900 ml 0.1 N HCl at 37° C.±0.5° C.

Yet in another embodiment, the dissolution of the present compositions may be simulated by in vitro studies in a dissolution apparatus with paddles at 50 rpm in 1000 ml 0.1 N HCl at 37° C.±0.5° C.

The present invention is about using an alternative to an ion pairing agent, stabilizing agent, and solubilizing agent in the compositions of methylnaltrexone and its salts and still producing an immediate release soluble composition that can achieve gastrointestinal absorption.

The present invention provides oral pharmaceutical compositions of methylnaltrexone particularly tablets and capsules of methylnaltrexone. The invention provides oral pharmaceutical compositions of methylnaltrexone bromide and ion exchange resin which forms methylnaltrexone-ion exchange resin complex which does not act as an ion pairing, solubilizing and stabilizing agent with methylnaltrexone and still produces an immediate release composition of methylnaltrexone. The resulting compositions are immediate release and release at least 60 to 90% methylnaltrexone in 15 minutes.

The present invention also relates to a process for preparation of oral stable composition of methylnaltrexone or its pharmaceutical acceptable salt thereof, wherein, said process comprises; (i) preparing a drug-resin complex comprising methylnaltrexone or a pharmaceutical acceptable salt thereof and a pharmaceutically acceptable ion exchange resins and one or more pharmaceutically acceptable excipients; (ii) sifting extragranular material and lubricant; (iii) mixing the obtained drug-resin complex with sifted extragranular material and lubricant followed by compression of the obtained blend with suitable tooling; (iv) applying coating composition on the obtained compressed blend.

The following examples are meant to illustrate the present invention and should not be construed as limiting its scope.

### EXAMPLES

### Example 1: Immediate Release Tablets Composition (1:0.1)

TABLE 1
Composition of methylnaltrexone bromide
and ion exchange resin in 1:0.1 ratio

Quantity per Tablet

| | | |
|---|---|---|
| Name of the ingredient | Wt. (mg) | % w/w |

Intragranular

| | | |
|---|---|---|
| Methylnaltrexone bromide | 150.000 | 28.11 |
| Sodium polystyrene sulfonate | 15.000 | 2.81 |
| (Amberlite IRP 69) | | |
| Purified water | Q.S. | |

Extragranular

| | | |
|---|---|---|
| Silicified microcrystalline | 299.600 | 56.15 |
| cellulose | | |
| Crospovidone | 25.000 | 4.69 |
| Croscarmellose sodium | 25.900 | 4.85 |
| Stearic acid | 2.500 | 0.47 |
| Core Tablet Weight (mg) | 518.000 | |

Film Coating

| | | |
|---|---|---|
| Opadry | 15.540 | 2.91 |
| Purified water | Q.S. | — |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate (Amberlite IRP 69) in step 2 dispersion under stirring for 2 hours.

Step 4. Dry the step 3 slurry in tray dryer to obtain NMT 2.5% w/w LOD.

Step 5. Sift the step 4 dried granules through 30 mesh s.s. sieve.

Step 6. Sift Extragranular material i.e., Silicified Microcrystalline Cellulose, Crospovidone and Croscarmellose Sodium through 30 mesh s.s. sieve.

Step 7. Sift extra granular Stearic Acid through 60 mesh s.s. sieve.

Step 8. Load half quantity of step 6 followed by step 5 and remaining quantity of step 6 material in bin blender. Mix for 15 minutes at 5 rpm.

Step 9. Add step 7 material to step 8 and mix for 3 minutes at 5 rpm.

Step 10. Compress the blend of step 9 using suitable tooling into tablet.

Step 11. Coat the tablet of step 10 using Opadry solution up to 3.0% w/w build up.

### Example 2: Immediate Release Tablet Composition (1:0.25)

TABLE 2
Composition Methylnaltrexone bromide
and ion exchange resin in1:0.25 ratio

Quantity per Tablet

| | | |
|---|---|---|
| Name of the ingredient | Wt. (mg) | % w/w |

Intragranular

| | | |
|---|---|---|
| Methylnaltrexone bromide | 150.000 | 28.11 |
| Sodium polystyrene sulfonate | 37.500 | 7.03 |
| (Amberlite IRP 69) | | |
| Purified water | Q.S. | |

Extragranular

| | | |
|---|---|---|
| Silicified microcrystalline | 277.100 | 51.94 |
| cellulose | | |
| Crospovidone | 25.000 | 4.69 |
| Croscarmellose sodium | 25.900 | 4.85 |
| Stearic acid | 2.500 | 0.47 |
| Core Tablet Weight (mg) | 518.000 | |

Film Coating

| | | |
|---|---|---|
| Opadry | 15.540 | 2.91 |
| Purified water | Q.S. | |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate (Amberlite IRP 69) in step 2 dispersion under stirring for 2 hours.

Step 4. Dry the step 3 slurry in tray dryer to obtain NMT 2.5% w/w LOD.

Step 5. Sift the step 4 dried granules through 30 mesh s.s. sieve.

Step 6. Sift Extragranular material i.e., Silicified Microcrystalline Cellulose, Crospovidone and Croscarmellose Sodium through 30 mesh s.s. sieve.

Step 7. Sift extra granular Stearic Acid through 60 mesh s.s. sieve.

Step 8. Load half quantity of step 6 followed by step 5 and remaining quantity of step 6 material in bin blender. Mix for 15 minutes at 5 rpm.

Step 9. Add step 7 material to step 8 and mix for 3 minutes at 5 rpm.

Step 10. Compress the blend of step 9 using suitable tooling into tablet.

Step 11. Coat the tablet of step 10 using Opadry solution up to 3.0% w/w build up.

### Example 3: Immediate Release Tablet Composition (1:0.5)

TABLE 3
Composition methylnaltrexone bromide
and ion exchange resin in1:0.5 ratio

Quantity per Tablet

| | | |
|---|---|---|
| Name of the ingredient | Wt. (mg) | % w/w |

Intragranular

| | | |
|---|---|---|
| Methylnaltrexone bromide | 150.000 | 28.11 |
| Sodium polystyrene sulfonate | 75.000 | 14.06 |
| (Amberlite IRP 69) | | |
| Purified water | Q.S. | |

Extragranular

| | | |
|---|---|---|
| Silicified microcrystalline | 226.075 | 42.37 |
| cellulose | | |
| Crospovidone | 25.000 | 4.69 |
| Croscarmellose sodium | 25.900 | 4.85 |
| Stearic acid | 2.500 | 0.47 |
| Core Tablet Weight (mg) | 518.000 | |

Extragranular

| | | |
|---|---|---|
| Opadry | 15.540 | 2.91 |
| Purified water | Q.S. | |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate (Amberlite IRP 69) in step 2 dispersion under stirring for 2 hours.

Step 4. Dry the step 3 slurry in tray dryer to obtain NMT 2.5% w/w LOD.

Step 5. Sift the step 4 dried granules through 30 mesh s.s. sieve.

Step 6. Sift Extragranular material i.e. Silicified Microcrystalline Cellulose, Crospovidone and Croscarmellose Sodium through 30 mesh s.s. sieve.

Step 7. Sift extra granular Stearic Acid through 60 mesh s.s. sieve.

Step 8. Load half quantity of step 6 followed by step 5 and remaining quantity of step 6 material in bin blender. Mix for 15 minutes at 5 rpm.

Step 9. Add step 7 material to step 8 and mix for 3 minutes at 5 rpm.

Step 10. Compress the blend of step 9 using suitable tooling into tablet.

Step 11. Coat the tablet of step 10 using Opadry solution up to 3.0% w/w build up.

### Example 4: Immediate Release Tablet Composition (1:1)

TABLE 4
Composition methylnaltrexone bromide
and ion exchange resin in 1:1 ratio

Quantity per Tablet

| | | |
|---|---|---|
| Name of the ingredient | Wt. (mg) | % w/w |

Intragranular

| | | |
|---|---|---|
| Methylnaltrexone bromide | 150.00 | 28.11 |
| Sodium polystyrene sulfonate | 150.00 | 28.11 |
| (Amberlite IRP 69) | | |
| Purified water | Q.S. | |

Extragranular

| | | |
|---|---|---|
| Silicified microcrystalline | 164.600 | 30.85 |
| cellulose | | |
| Crospovidone | 25.000 | 4.69 |
| Croscarmellose sodium | 25.900 | 4.85 |
| Stearic acid | 2.500 | 0.47 |
| Core Tablet Weight (mg) | 518.000 | |

Film coatings

| | | |
|---|---|---|
| Opadry | 15.540 | 2.91 |
| Purified water | Q.S. | |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate (Amberlite IRP 69) in step 2 dispersion under stirring for 2 hours.

Step 4. Dry the step 3 slurry in tray dryer to obtain NMT 2.5% w/w LOD.

Step 5. Sift the step 4 dried granules through 30 mesh s.s. sieve.

Step 6. Sift Extragranular material i.e., Silicified Microcrystalline Cellulose, Crospovidone and Croscarmellose Sodium through 30 mesh s.s. sieve.

Step 7. Sift extra granular Stearic Acid through 60 mesh s.s. sieve.

Step 8. Load half quantity of step 6 followed by step 5 and remaining quantity of step 6 material in bin blender. Mix for 15 minutes at 5 rpm.

Step 9. Add step 7 material to step 8 and mix for 3 minutes at 5 rpm.

Step 10. Compress the blend of step 9 using suitable tooling into tablet.

Step 11. Coat the tablet of step 10 using Opadry solution up to 3.0% w/w build up.

### Example 5: In Vitro Release of Example 1 to 4

TABLE 6
Dissolution of batches explained in example 1 to
4 performed in paddle apparatus at 50 RPM in 1000
mL 0.1N HCl at 37° C. ± 0.5° C.

| | | Example 1 | Example 2 | Example 3 | Example 4 |
|---|---|---|---|---|---|
| | Time | (1:0.1) % | (1:0.25) % | (1:0.5) % | (1:1) % |
| Media | (min.) | Release | Release | Release | Release |
| Paddle | 5 | 87 | 89 | 82 | 63 |
| 50 RPM | 10 | 93 | 92 | 85 | 65 |
| 1000 mL | 15 | 96 | 93 | 86 | 66 |
| 0.1N HCl | 20 | 98 | 94 | 87 | 68 |
| at 37° C. ± | 30 | 100 | 94 | 89 | 70 |
| 0.5° C. | 45 | 101 | 95 | 91 | 71 |
| | 60 | 102 | 95 | 91 | 72 |

### Example 6: Immediate Release Tablets Composition (1:0.05)

TABLE 6
Composition methylnaltrexone bromide and ion exchange
resin in1:0.05 ratio with sodium chloride

Quantity per Tablet in mg

| | | |
|---|---|---|
| Name of the ingredient | 150 mg | % w/w |

Intragranular

| | | |
|---|---|---|
| Methylnaltrexone bromide | 150.000 | 28.114 |
| Sodium polystyrene sulfonate | 7.500 | 1.405 |
| (Amberlite IRP 69) | | |
| Purified water | Q.S. | — |

Extragranular

| | | |
|---|---|---|
| Silicified microcrystalline | 297.100 | 55.685 |
| Crospovidone | 25.000 | 4.686 |
| Croscarmellose sodium | 25.900 | 4.854 |
| Sodium chloride | 10.000 | 1.874 |
| Stearic acid | 2.500 | 0.469 |
| Core Tablet Weight | 518.000 | |

Film Coating

| | | |
|---|---|---|
| Opadry | 15.540 | 2.913 |
| Purified water | Q.S. | |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate in step 2 dispersion under stirring for 2 hours.

Step 4. Dry the step 3 slurry in tray dryer to obtain NMT 3.0% w/w LOD.

Step 5. Sift the step 4 dried granules through 30 mesh s.s. sieve.

Step 6. Sift Sodium Chloride through 80 mesh s.s. sieve.

Step 7. Sift Extragranular material i.e. Silicified Microcrystalline Cellulose, Crospovidone and Croscarmellose Sodium through 30 mesh s.s. sieve.

Step 8. Sift extra granular Stearic Acid through 60 mesh s.s. sieve.

Step 9. Load half quantity of step 6 and 7 followed by step 5 and the remaining quantity of step 6 and 7 materials in bin blender. Mix for 15 minutes at 5 rpm.

Step 10. Add step 7 material to step 8 and mix for 3 minutes at 5 rpm.

Step 11. Compress the blend of step 9 using suitable tooling into tablet.

Step 12. Coat the tablet of step 10 using Opadry solution up to 3.0% w/w build up.

### Example 7: Composition Methylnaltrexone Bromide and Ion Exchange Resin in 1:0.075 Ratio with Sodium Chloride

TABLE 7
Composition methylnaltrexone bromide and ion exchange
resin in 1:0.075 ratio with sodium chloride

Quantity per Tablet in mg

| | | |
|---|---|---|
| Name of the ingredient | 150 mg | % w/w |

Intragranular

| | | |
|---|---|---|
| Methylnaltrexone bromide | 150.000 | 28.114 |
| Sodium polystyrene sulfonate | 11.250 | 2.109 |
| (Amberlite IRP 69) | | |
| Purified water | Q.S. | — |

Extragranular

| | | |
|---|---|---|
| Silicified microcrystalline | 293.350 | 54.981 |
| Crospovidone | 25.000 | 4.686 |
| Croscarmellose sodium | 25.900 | 4.854 |
| Sodium chloride | 10.000 | 1.874 |
| Stearic acid | 2.500 | 0.469 |
| Core Tablet Weight (mg) | 518.000 | |

Film Coating

| | | |
|---|---|---|
| Opadry | 15.540 | 2.913 |
| Purified water | Q.S. | |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate in step 2 dispersion under stirring for 2 hours.

Step 4. Dry the step 3 slurry in tray dryer to obtain NMT 3.0% w/w LOD.

Step 5. Sift the step 4 dried granules through 30 mesh s.s. sieve.

Step 6. Sift Sodium Chloride through 80 mesh s.s. sieve.

Step 7. Sift Extragranular material i.e., Silicified Microcrystalline Cellulose, Crospovidone and Croscarmellose Sodium through 30 mesh s.s. sieve.

Step 8. Sift extra granular Stearic Acid through 60 mesh s.s. sieve.

Step 9. Load half the quantity of step 6 and 7 followed by step 5 and remaining quantity of step 6 and 7 materials in bin blender. Mix for 15 minutes at 5 rpm.

Step 10. Add step 7 material to step 8 and mix for 3 minutes at 5 rpm.

Step 11. Compress the blend of step 9 using suitable tooling into tablet.

Step 12. Coat the tablet of step 10 using Opadry solution up to 3.0% w/w build up.

### Example 8: Composition Methylnaltrexone Bromide and Ion Exchange Resin in 1:0.1 Ratio with Sodium Chloride

TABLE 8
Composition methylnaltrexone bromide and ion exchange
resin in 1:0.1 ratio with sodium chloride

Quantity per Tablet in mg

| | | |
|---|---|---|
| Name of the ingredient | 150 mg | % w/w |

Intragranular

| | | |
|---|---|---|
| Methylnaltrexone bromide | 150.000 | 28.114 |
| Sodium polystyrene sulfonate | 15.000 | 2.811 |
| (Amberlite IRP 69) | | |
| Purified Water | Q.S. | |

Extragranular

| | | |
|---|---|---|
| Silicified microcrystalline | 289.600 | 54.279 |
| Crospovidone | 25.000 | 4.686 |
| Croscarmellose sodium | 25.900 | 4.854 |
| Sodium chloride | 10.000 | 1.874 |
| Stearic acid | 2.500 | 0.469 |
| Core Tablet Weight | 518.000 | |

Film Coating

| | | |
|---|---|---|
| Opadry | 15.540 | 2.913 |
| Purified water | Q.S. | |
| Coated Tablet Weight (mg) | 533.540 | 100 |

### Manufacturing Procedure:

Step 1. Check and verify the weights and AR. No.'s of dispensed ingredients. All the sieves used for sifting are ASTM.

Step 2. Disperse Methylnaltrexone Bromide USP in purified Water under stirring.

Step 3. Add Sodium Polystyrene sulfonate in step 2 dispersion under stirring for 2 hours.

Step 4. Dry the step 3 slurry in tray dryer to obtain NMT 3.0% w/w LOD.

Step 5. Sift the step 4 dried granules through 30 mesh s.s. sieve.

Step 6. Sift Sodium Chloride through 80 mesh s.s. sieve.

Step 7. Sift Extragranular material i.e., Silicified Microcrystalline Cellulose, Crospovidone and Croscarmellose Sodium through 30 mesh s.s. sieve.

Step 8. Sift extra granular Stearic Acid through 60 mesh s.s. sieve.

Step 9. Load half quantity of step 6 and 7 followed by step 5 and the remaining quantity of step 6 and 7 materials in bin blender. Mix for 15 minutes at 5 rpm.

Step 10. Add step 7 material to step 8 and mix for 3 minutes at 5 rpm.

Step 11. Compress the blend of step 9 using suitable tooling into tablet.

Step 12. Coat the tablet of step 10 using Opadry solution up to 3.0% w/w build up. Stability Studies:

Tablet composition in accordance with example 1 containing 150 mg of methylnaltrexone bromide, packed in 50 cc HDPE bottle with CR closure. The pack was subjected to storage stability condition such as 25° C./60% RH for three months. The pack was also subjected to accelerated storage conditions of 40° C./75% RH for a period of three months. The tablets were analyzed for water content, in-vitro dissolution, related substances or degradation products and assay.

TABLE 9
In vitro release, % assay, water content, and degradation impurity of
150 mg of methylnaltrexone tablets of example 1 packaged in 50 cc HDPE
bottle with CR closure after 1, 3 month accelerated stability condition
of 40° C./75% RH and 3-month stability condition of 25° C./60% RH.

| | Storage Condition | | | | |
|---|---|---|---|---|---|
| | | 40° C./ | 25° C./ | 40° C./ | |
| | NA | 75% RH | 60% RH | 75% RH | |
| | Tests | | | | |
| | Initial | 1 M | 3 M | 3 M | Limit |
| Water content by KF | 3.75 | 4.53 | 4.12 | 4.20 | NMT 7.0 |
| (% w/w) | | | | | |

Dissolution: % in vitro release of methylnaltrexone in 1000 mL,
0.1N HCl, USP Type-II (Paddle), 50 RPM at temp. 37° C. ± 0.5° C.

| Time | Initial | 1 M | 3 M | 3 M | Limit |
|---|---|---|---|---|---|
| 5 Min | 84 | 76 | 92 | 92 | NLT 80 % (Q) |
| 10 Min | 89 | 85 | 97 | 95 | of the labelled |
| 15 Min | 91 | 87 | 94 | 97 | amount of |
| 20 Min | 92 | 88 | 98 | 98 | methylnaltrexone |
| 30 Min | 94 | 90 | 98 | 97 | dissolved in 60 |
| 45 Min | 97 | 93 | 99 | 97 | minutes |
| 60 Min | 98 | 94 | 98 | 98 | |

Assay (%)

| Each Tablet Contains | | | | | |
|---|---|---|---|---|---|
| Methylnaltrexone | Initial | 1 M | 3 M | 3 M | Limit |
| mg/Tablet | 151.30 | 146.63 | 149.86 | 154.63 | 135.00 mg to |
| | | | | | 165.00 mg |
| % Label Claim | 100.9 | 97.8 | 99.9 | 103.1 | 90.0 to 110.0% |
| | | | | | of label claim |

Degradation Product by HPLC (%)

| — | Initial | 1 M | 3 M | 3 M | Limit |
|---|---|---|---|---|---|
| S-Methylnaltrexone | ND | BDL | BDL (0.014) | BDL (0.015) | NMT 0.2% |
| Naltrexone | BDL | BDl | BDL (0.009) | BDL (0.004) | NMT 0.2% |
| N-Butenyl oxymorphone | ND | BDL | BDL (0.01) | BDL (0.013) | NMT 0.2% |
| Acetylmethylnaltrexone | ND | ND | ND | ND | NMT 0.2% |
| Acetylnaltrexone | ND | BDL | ND | ND | NMT 0.2% |
| O-Methyl methylnaltrexone | BDL | ND | BQL (0.036) | BQL (0.038) | NMT 0.2% |
| Any Unspecified | 0.05 | 0.1 | 0.06 | 0.05 | NMT 0.2% |
| Degradation Product | | (RRT 1.05) | (RRT 0.79) | (RRT 0.79) | |
| Total Degradation Products | 0.18 | 0.24 | 0.14 | 0.13 | NMT 1.0% |

The stability data in table 9 reveals that there is no significant change in the in-vitro dissolution, related substances/degradation products and assay value, of methylnaltrexone tablets, in HDPE bottles with CR closure on storage. The said tablets were found to be stable for a period of at least 3 months at 25° C./60% RH condition and accelerated storage stability condition 40° C./75% RH.

1. A stable pharmaceutical composition comprising (i) methylnaltrexone or its pharmaceutically acceptable salt thereof; (ii) ion exchange resin particles; and at least one or more excipient.

2. The composition according to claim 1, wherein said composition is suitable for oral administration.

3. The composition according to claim 1, wherein said composition comprises about 50 mg to about 500 mg or from about 150 mg to about 450 mg dose of methylnaltrexone bromide.

4. The composition according to claim 1, wherein said composition is administered for treatment of opioid-induced constipation in adult patients with cancer and chronic non-cancer pain.

5. The composition according to claim 1, wherein said composition is administered once daily for treatment of opioid-induced constipation in adult patients with cancer and chronic non-cancer pain.

6. The composition according to claim 1, wherein said composition is in the solid dosage form.

7. The composition according to claim 6, wherein the said solid dosage form comprises tablets, capsules, dispersible tablets, chewable tablets, granules, spheres, powder, multiunit particulate systems (MUPS), beads, and particles.

8. The composition according to claim 1, wherein said ion exchange resin particles are selected from the group comprising of anionic resins such as DUOLITE® AP143/1083 (cholestyramine resin USP), cationic resins such as AMBERLITE® IRP-64 (a porous copolymer of methacrylic acid crosslinked with divinylbenzene), AMBERLITE® IRP-69 (Sodium polystyrene sulfonate USP) and AMBERLITE® IRP-88.

9. The composition according to claim 1, wherein methylnaltrexone or its acceptable salt is complexed with ion-exchange resin particles to obtain methylnaltrexone ion exchange resin complex.

10. The composition according to claim 1, wherein said composition and/or methylnaltrexone ion exchange resin complex comprises methylnaltrexone or its pharmaceutically acceptable salt and ion exchange resin particles in a ratio from about 1:0.01 to about 1:10, or from about 1:0.05 to about 1:1.

11. The composition according to claim 1, wherein at least one or more excipient comprises one or more diluent or filler selected from calcium phosphate, dicalcium phosphate, tricalcium phosphate, calcium sulfate, anhydrous lactose, spray dried lactose, hydrated lactose, cellulose, spray dried microcrystalline cellulose, spray dried combinations comprising microcrystalline cellulose and lactose, silicified microcrystalline cellulose, kaolin, bentonite, mannitol, starch, magnesium carbonate, sorbitol, sucrose, inositol, compressible sugar, trehalose and xylitol, and mixtures thereof.

12. The composition according to claim 1, wherein at least one or more excipient comprises diluent or filler from about 10 to about 90%, or from about 15 to about 85%, or from about 20 to about 75%, or from about 30 to about 70%, or from about 15 to about 30%, or from about 50 to about 90%, or from about 60 to about 85%.

13. The composition according to claim 1, wherein at least one or more excipient comprises one or more disintegrant is selected from crospovidone, sodium croscarmellose and/or sodium starch glycolate, pregelatinized starch, low substituted hydroxypropyl cellulose (L-HPC).

14. The composition according to claim 1, wherein at least one or more excipient comprises disintegrant from about 3 to about 20%, or from about 5 to about 20%, or from about 5 to about 15%.

15. The composition according to claim 1, wherein at least one or more excipient comprises one or more osmotic agent is selected from sucrose, xylitol, glucose, lactose, salts such as sodium chloride, potassium chloride; low molecular weight hydrophilic polymers such as cellulose ethers, maltodextrins, and cyclodextrins.

16. The composition according to claim 1, wherein at least one or more excipient comprises osmotic agent from about 0.1 to about 10%, or from about 0.1 to about 5%.

17. The composition according to claim 1, wherein at least one or more excipient comprises one or more lubricant selected from magnesium stearate, stearic acid, palmitic acid, calcium stearate, talc, carnauba wax and sodium stearyl fumarate.

18. The composition according to claim 1, wherein at least one or more excipient comprise lubricant from about 0.1 to about 5% by weight.

19. The composition according to claim 1, wherein said composition is an immediate release composition.

20. The composition according to claim 1, wherein said composition releases about 60%, about 75%, about 80%, about 85%, about 90%, about 95%, or about 99% of the methylnaltrexone in 15 minutes when measured using the USP type II paddle apparatus with 50 rpm in 1000 mL of 0.IN HCL at temp. 37° C.±0.5° C.

21. The composition according to claim 1, wherein said composition is substantially free of impurities.
